(19) United States
(12) Patent Application Publication
(10) Pub. No.: US 2011/0236491 A1
(43) Pub. Date: Sep. 29, 2011

(54) TOPICAL ANTI-INFLAMMATORY COMPOSITION

(76) Inventors: Jeannette Chantalat, Pennington, NJ (US); Jue-Chen Liu, Belle Mead, NJ (US); May Shana'a, Martinsville, NJ (US); Michael Southall, Lawrenceville, NJ (US); Ying Sun, Belle Mead, NJ (US)

(21) Appl. No.: 12/842,661

(22) Filed: Jul. 23, 2010

Related U.S. Application Data
(63) Continuation-in-part of application No. 12/680,105, filed on Mar. 25, 2010.

(51) Int. Cl. A61K 38/43; A61K 9/14; A61K 33/30; A61K 33/34; A61K 33/38; A61P 29/00

(52) U.S. Cl. 424/490; 424/94.1; 424/641; 424/630; 424/618

(57) ABSTRACT

Compositions and methods for treating inflammation in mammalian tissue are provided using substantially continuously, in situ generated, anti-inflammatory effective amounts of hydrogen peroxide.

### FIELD OF THE INVENTION

Compositions and methods for treating inflammation in mammalian tissue are provided using substantially continuously, in situ generated, anti-inflammatory effective amounts of hydrogen peroxide. The hydrogen peroxide may be generated by electrochemical reaction, photochemical reaction, catalytic reaction, enzymatic reaction, or combinations thereof.

### BACKGROUND OF THE INVENTION

The skin ages in two key and distinct ways. Intrinsic skin aging is closely linked with one's genetic constitution, which is unchangeable and inherited. Possessing the right combination and number of “health” genes varies from person to person. Intrinsic aging occurs chronologically as the body's processes slow down and skin cells are not replaced as rapidly as they once were. This leads to a reduction in the production of new collagen and elastin in the skin, and wrinkles and lines are more prominent as a result of this deterioration of the skin's fibrous structure. A thinning of the epidermis also causes a loss of barrier properties, further contributing to dehydration and increasing the visible signs of aging. Extrinsic skin aging occurs both through environmental exposure to the sun, toxins and pollution, and due to poor personal habits such as poor diet or smoking. Taken together, extrinsic skin aging can accelerate the intrinsic skin aging process, leading to a prematurely aged look.

A key process involved in skin aging is inflammation. Although inflammation is generally thought to involve visible signs such as swelling, pain and redness, sub-clinical inflammation, meaning inflammation below the threshold of visually clinical detection, also cues our body to injury and danger occurring from within. Recent studies have demonstrated that sub-clinical inflammation can be detected using sensitive biochemical techniques, such as measuring increased levels of pro-inflammatory proteins in skin, even though the skin does not show a visible redness or swelling. Garay et al., Journal of the American Academy of Dermatology, Volume 60, Issue 3, Supplement 1, Page AB28, March 2009.

During intrinsic aging of the skin, free radicals and other chemicals are released from aging cells as normal, ongoing processes. Chronic release of these chemicals induces sub-clinical inflammation in the skin, which drives the breakdown in the structure and function of the skin. During extrinsic aging, inflammation is more apparent.

Inflammation also plays an important role in tissue healing and repairing. While some tissue inflammation in response to cell damage, irritants, or pathogens is beneficial for healing, excess inflammation is known to hinder the healing and repairing processes. Because most of anti-inflammatory drugs (such as non-steroidal anti-inflammatory drugs or NSAIDS) tend to retard healing, their use is discouraged during in the early phase of post-surgery situations. See for example, Ferry, et al., The American Journal of Sports Medicine, Vol. 35, No. 8, 2007, pages 1326-1333). It is therefore highly desirable to be able to modulate or reduce excessive tissue inflammation without retardation of tissue healing and repairing process.

Hydrogen peroxide (H₂O₂) is a very pale blue liquid that appears colorless in a dilute solution, slightly more viscous than water. It has strong oxidizing properties and is therefore a powerful bleaching agent that has found use as a disinfectant. Hydrogen peroxide is an effective anti-bacterial, anti-fungal, and anti-viral compound that is even effective against methicillin resistant Staphylococcus aureus (MRSA) isolates. In addition, rinsing the oral cavity with a solution of hydrogen peroxide was reported to result in a significant reduction of aerobic and anaerobic bacteria in saliva. The reduction in bacteria in the oral cavity can help reduce the incidence of gingivitis. Peroxides have been used in tooth whitening for more than 100 years and hydrogen peroxide is one of the most commonly used active ingredients used in tooth whitening. Hydrogen peroxide is also an effective vasoconstrictor which can reduce the appearance of dark circles, and result in a skin whitening effect. Stamatas et al., 2004 J Biomed Opt. 9:315-322.

However, workers in the art have also found that certain levels of hydrogen peroxide produce detrimental effects in humans. See for instance, Miyoshi et al., PNAS, Vol. 103, No. 6, pages 1727-1731 (Feb. 7, 2006) and Mastrangelo et al., Am. Occ. Hyg., Oxford University Press, pages 1-5 (2008). Miyoshi et al. found human fibroblasts exposed to 20 μM, 30 μM and 40 μM of hydrogen peroxide showed increasing losses of cell viability that was exacerbated by age.

U.S. Pat. Nos. 6,673,374, 6,475,472, 6,383,523, and 7,018,660 disclose a topical anti-inflammatory pharmaceutical composition that includes, among other ingredients, hydrogen peroxide in an amount sufficient to cleanse the skin and a separate anti-inflammatory agent. The hydrogen peroxide is present in an amount from about 0.01 to 6 weight percent by weight of the composition.

US Published Appln. No. 2009/0304811 relates to a formulation especially useful for anti-microbial, ophthalmic formulations that comprises a polysaccharide and a source of hydrogen peroxide, such as hydrogen peroxide itself, urea hydrogen peroxide, or perborate salts. The hydrogen peroxide generated is in the range of about 0.0001 to about 5 weight percent of the formulation. However, it is produced via chemical reaction all at once, i.e., not continuously.

It is known in the art, and acknowledged in US 2009/0304811, that hydrogen peroxide decomposes very easily, especially in the presence of other compounds. Accordingly, it would be highly desirable to have a composition that is both stable and enables the administration of hydrogen peroxide on a continuous basis.

It has now been discovered that a low, anti-inflammatory effective amount of hydrogen peroxide may be generated in situ in mammalian tissue on a substantially continuously and sustained basis. When the tissue is skin, such administration of hydrogen peroxide provides the specific benefit of reduced subclinical inflammation and, in turn, enhanced production of elastin and collagen.

Commercially available hydrogen peroxide purchased over the counter for anti-septic purposes typically contains 3% hydrogen peroxide. In contrast, it has now been found that on the order of 1/30000 of such amount of hydrogen peroxide advantageously generates an anti-inflammatory effect in mammalian tissues.

### SUMMARY OF THE INVENTION

This invention provides a method of treating inflammation in a mammalian tissue comprising administering to said tissue a composition capable of substantially continuously generating an anti-inflammatory effective amount of hydrogen peroxide in situ.

The invention also provides a method of substantially continuously generating hydrogen peroxide in situ on a mammalian tissue, which comprises contacting said tissue with a composition comprising a hydrogen peroxide generating agent selected from the group consisting of galvanic particulates, vitamins, metal oxides, enzymes and their substrates, and combinations thereof.

The invention also provides an anti-inflammatory composition capable of substantially continuously generating an anti-inflammatory effective amount of hydrogen peroxide in mammalian tissues in situ.

### DETAILED DESCRIPTION OF THE INVENTION

It is believed that one skilled in the art can, based upon the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. Unless otherwise indicated, a percentage refers to a percentage by weight (i.e., % (W/W)). Unless stated otherwise, all ranges are inclusive of the endpoints, e.g., “from 4 to 9” includes the endpoints 4 and 9.

As used herein, the term “continuous” release means release at a steady, controlled rate. Release of hydrogen peroxide does not occur sporadically or in an unpredictable fashion, or in a “burst” release fashion. The continuous release may be steady state (commonly referred to as “timed release” or zero-order drug release kinetics) such that the hydrogen peroxide is released in even amounts over a predetermined time.

As used herein, the term “anti-inflammatory effective amount” means an amount of hydrogen peroxide sufficient to treat subclinical inflammation, clinical inflammation, or both. Preferably, the anti-inflammatory effective amount is less than about 0.5 weight percent of the composition from which the hydrogen peroxide is generated. In one embodiment, the anti-inflammatory effective amount of hydrogen peroxide is about 0.0005 to about 0.5% by weight of the composition. More preferably, the anti-inflammatory effective amount is less than about 0.1% by weight of the composition. Even more preferably, anti-inflammatory effective amount is about 0.001% to about 0.05%, and most preferably about 0.005% to about 0.05% by weight of the composition.

As used herein, the term “pharmaceutically-acceptable,” “dermatologically-acceptable,” or cosmetically-acceptable” means that the ingredients which the term describes are suitable for use in contact with mammalian tissue (e.g., the skin or mucosa) without undue toxicity, incompatibility, instability, irritation, allergic response, and the like.

As used herein, the term “safe and effective” means sufficient to provide the desired benefit at a desired level, but low enough to avoid serious side effects. The safe amount of the ingredient or composition will vary with the area being treated, the age and tissue of the patient, the duration and nature of the treatment, the specific ingredients or composition employed, the particular carrier utilized, and like factors.

As used herein, the terms “treat” or “treatment” means the treatment (e.g., alleviation or elimination of symptoms and/or cure) and/or prevention or inhibition of a disease or condition.

As used herein, “mammalian tissue” means tissue of a human or other mammal, including internal tissues (muscle, nerve, bone and connective tissues), external tissues such as barrier membranes, or mucosal membranes, such as oral, rectal, or vaginal musocal membranes. Mammalian tissue includes soft tissues (e.g., the skin, mucosa, epithelium, wound, eye and its surrounding tissues, cartilage and other soft musculoskeletal tissues such as ligaments, tendons, or meniscus), hard tissues (e.g., bone, teeth, nail matrix, or hair follicle), and soft tissue-hard tissue conjunctions (e.g., conductive tissues around periodontal area involved teeth, bones or soft tissue of the joint).

As used herein, the term “barrier membrane” means the thin layer of tissue which covers a surface thereby separating cellular structures or organs. Barrier membrane includes, without limitation, epidermis or epithelial tissue. As used herein, the term “skin” means all external surfaces of a patient, such as the exposed hide or surfaces covered by hair.

The term “patient” refers to a mammal which is being treated. Preferably the patient is a human.

As used herein, the terms “inflammatory disorders” and “inflammation” generally mean a reaction of mammalian tissue to irritation, infection, or injury. “Clinical inflammation” can appear as visible redness (erythema), swelling (edema), or as a bruise (contusion). “Subclinical inflammation” refers to the phase of inflammation prior to the manifestation of visible symptoms. Subclinical inflammation is a low level of inflammation characterized by an elevated level of free radicals and pro-inflammatory proteins.

Inflammatory disorders and related conditions include, but are not limited to, arthritis, bronchitis, contact dermatitis, atopic dermatitis, psoriasis, seborrheic dermatitis, eczema, allergic dermatitis, polymorphous light eruptions, inflammatory dermatoses, folliculitis, alopecia, poison ivy, insect bites, acne inflammation, rosacea inflammation, skin or mucosal condition of irritation, edma, itch or pain. Specifically, the inflammatory disorders and related conditions are arthritis, inflammatory dermatoses, contact dermatitis, allergic dermatitis, atopic dermatitis, polymorphous light eruptions, irritation, including erythema induced by extrinsic factors, acne inflammation, psoriasis, seborrheic dermatitis, eczema, poison ivy, insect bites, folliculitus, alopecia, and secondary conditions and the like. Secondary conditions resulting from inflammation include, but not limited to, xerosis, hyperkeratosis, pruritus, post-inflammatory hyperpigmentation, scarring and the like.

In one aspect, the invention provides a composition capable of substantially continuously generating an anti-inflammatory effective amount of hydrogen peroxide in mammalian tissues in situ. The composition may comprise an agent capable of generating such hydrogen peroxide via electrochemical reaction, photochemical reaction, catalytic reaction, enzymatic reaction, or a combination thereof.

### Hydrogen Peroxide Produced by Electrochemical Reaction

In one embodiment, the hydrogen peroxide generating agent comprises galvanic particulates. Galvanic particulates are capable of generating hydrogen peroxide substantially continuously on a mammalian tissue in situ via an electrochemical reaction (described more fully below).

The galvanic particulates comprise a first conductive material and a second conductive material, wherein both the first conductive material and the second conductive material are exposed on the surface of the galvanic particulate. In one embodiment, the galvanic particulates comprise the first conductive material partially coated with the second conductive material.

In one embodiment, the galvanic particulates are produced by a coating method wherein the weight percentage of the second conductive material is from about 0.001% to about 20%, by weight, of the total weight of the particulate, such as from about 0.01% to about 10%, by weight, of the total weight of galvanic particulate. In one embodiment, the coating thickness of the second conductive material may vary from single atom up to hundreds of microns. In yet another embodiment, the surface of the galvanic particulate comprises from about 0.001 percent to about 99.99 percent such as from about 0.1 to about 99.9 percent of the second conductive material.

In one embodiment, the galvanic particulates are fine enough that they can be suspended in the semi-solid compositions during storage. In a further embodiment, they are in flattened and/or elongated shapes. The advantages of flattened and elongated shapes of the galvanic particulates include a lower apparent density and, therefore, a better floating/suspending capability in the topical composition, as well as better coverage over the biological tissue, leading to a wider and/or deeper range of the galvanic current passing through the biological tissue (e.g., the skin or mucosa membrane). In one embodiment, the longest dimension of the galvanic particulates is at least twice (e.g., at least five times) the shortest dimension of such particulates.

In one embodiment, the galvanic particulate comprises at least 90 percent, by weight, of conductive materials (e.g., the first conductive material and the second conductive material), such as at least 95 percent, by weight, or at least 99 percent, by weight, when a coating method is used for the production of the galvanic particulates.

Examples of combinations of first conductive materials and second conductive materials include (with a “/” sign representing an oxidized but essentially non-soluble form of the metal), but are not limited to, zinc-copper, zinc-copper/copper halide, zinc-copper/copper oxide, magnesium-copper, magnesium-copper/copper halide, zinc-silver, zinc-silver/silver oxide, zinc-silver/silver halide, zinc-silver/silver chloride, zinc-silver/silver bromide, zinc-silver/silver iodide, zinc-silver/silver fluoride, zinc-gold, zinc-carbon, magnesium-gold, magnesium-silver, magnesium-silver/silver oxide, magnesium-silver/silver halide, magnesium-silver/silver chloride, magnesium-silver/silver bromide, magnesium-silver/silver iodide, magnesium-silver/silver fluoride, magnesium-carbon, aluminum-copper, aluminum-gold, aluminum-silver, aluminum-silver/silver oxide, aluminum-silver/silver halide, aluminum-silver/silver chloride, aluminum-silver/silver bromide, aluminum-silver/silver iodide, aluminum-silver/silver fluoride, aluminum-carbon, copper-silver/silver halide, copper-silver/silver chloride, copper-silver/silver bromide, copper-silver/silver iodide, copper-silver/silver fluoride, iron-copper, iron-copper/copper oxide, copper-carbon iron-copper/copper halide, iron-silver, iron-silver/silver oxide, iron-silver/silver halide, iron-silver/silver chloride, iron-silver/silver bromide, iron-silver/silver iodide, iron-silver/silver fluoride, iron-gold, iron-conductive carbon, zinc-conductive carbon, copper-conductive carbon, magnesium-conductive carbon, and aluminum-carbon. When the first conductive and the second conductive materials are elemental metals (e.g., galvanic particulates of zinc-copper, zinc-silver, magnesium-copper, magnesium-silver, which are preferred galvanic particulates in the present invention), the first conductive metals are oxidizable metals (i.e., with high oxidation potential, or low reduction potential such as zinc and magnesium), and the second conductive metals are reducible metals (i.e., with low oxidation potential or high reduction potential such as copper and silver).

The first conductive material or second conductive material may also be alloys, particularly the first conductive material. Non-limiting examples of the alloys include alloys of zinc, iron, aluminum, magnesium, copper and manganese as the first conductive material and alloys of silver, copper, stainless steel and gold as second conductive material.

In another embodiment, the galvanic particulate can comprise a plurality of conductive materials or metals, namely, the number can be greater than 2 (binary) or 3 (tertiary). A non-limiting example of such a galvanic particulate can have the composition of magnesium-zinc-iron-copper-silver-gold in the form of multiple coatings or multiple conductive metal composite.

In one embodiment, the galvanic particulate, made of the first conductive material, is partially coated with several conductive materials, such as with a second and third conductive material. In a further embodiment, the particulate comprises at least 95 percent, by weight, of the first conductive material, the second conductive material, and the third conductive material. In one embodiment, the first conductive material is zinc, the second conductive material is copper, and the third conductive material is silver.

In one embodiment, the average particle size of the galvanic particulates ranges from about 10 nanometers to about 500 micrometers, preferably from about 100 nanometers to about 100 micrometers, and more preferably form about 1 micrometer to about 50 micrometers. What is meant by the particle size the maximum dimension measured in at least one direction of the particulates. The smaller the metal particles, the greater the galvanic reaction rate, hence more hydrogen peroxide can be generated.

In one embodiment, the galvanic particulates can be any shapes, such as spherical, oblong, flake, rod, needle, and irregular shape. These particulates can be individual particles or aggregates, or as a coating on a metallic or non-metallic substrate or particles.

In one embodiment, the difference in the Standard Electrode Potentials (or simply, Standard Potentials) of the first conductive material and the second conductive material is at least about 0.1 volts, such as at least 0.2 volts. In one embodiment, the materials that make up the galvanic couple have a Standard Potential difference equal to or less than about 3 volts. For example, for a galvanic couple comprised of metallic zinc and copper, the Standard Potential of zinc is −0.763V (Zn/Zn2⁺), and the Standard Potential of copper is +0.337 (Cu/Cu2⁺), and the difference in Standard Potentials is therefore 1.100V for the zinc-copper galvanic couple. Similarly, for a magnesium-copper galvanic couple, the Standard Potential of magnesium (Mg/Mg2⁺) is −2.363V, and the difference in the Standard Potentials is therefore 2.700V. Additional examples of Standard Potential values of some materials suitable for use in galvanic particulates are: Ag/Ag⁺: +0.799V, Ag/AgCl/Cl⁻: 0.222V, and Pt/H₂/H⁺: 0.000V. Pt may also be replaced by carbon or another conductive material. See, e.g., Physical Chemistry by Gordon M. Barrow, 4^{th} Ed., McGraw-Hill Book Company, 1979, page 626.

In one embodiment, the first and second conductive electrodes are combined (e.g., the second conductive electrode is deposited to the first conductive electrode) by chemical, electrochemical, physical or mechanical process (such as electroless deposition, electric plating, vacuum vapor deposition, arc spray, sintering, compacting, pressing, extrusion, printing, and granulation) conductive metal ink (e.g., with polymeric binders), or other known metal coating or powder processing methods commonly used in powder metallurgy, electronics or medical device manufacturing processes, such as the methods described in the book Asm Handbook Volume 7: Powder Metal Technologies and Applications (Asm International Handbook Committee, edited by Peter W. Lee, 1998, pages 31-109, 311-320). In another embodiment, all the conductive electrodes are manufactured by chemical reduction processes (e.g., electroless deposition), sequentially or simultaneously, in the presence of reducing agent(s). Examples of reducing agents include phosphorous-containing reducing agents (e.g., a hypophosphite as described in U.S. Pat. Nos. 4,167,416 and 5,304,403), boron-containing reducing agents, and aldehyde- or keton-containing reducing agents such as sodium tetrahydridoborate (NaBH₄) (e.g., as described in US 20050175649).

In one embodiment, the second conductive electrode is deposited or coated onto the first conductive electrode by physical deposition, such as spray coating, plasma coating, conductive ink coating, screen printing, dip coating, metals bonding, bombarding particulates under high pressure-high temperature, fluid bed processing, or vacuum deposition.

In one embodiment, the coating method is based on displacement chemical reaction, namely, contacting particles of the first conductive material (e.g., metallic zinc particles) with a solution containing a dissolved salt of the second conductive material (e.g. copper acetate, copper lactate, copper gluconate, or silver nitrate). In a further embodiment, the method includes flowing the solution over particles of the first conductive material (e.g., zinc powder) or through a packed powder of the first conductive material. In one embodiment, the salt solution is an aqueous solution. In another embodiment, the solution contains an organic solvent, such as an alcohol, a glycol, glycerin or other commonly used solvents in pharmaceutical production to regulate the deposition rate of the second conductive material onto the surfaces of the first conductive material particles, therefore controlling the activity of the galvanic particulates produced.

In another embodiment, the galvanic particulates of the present invention may also be coated with other materials to protect the first and second conductive materials from degradation during storage (e.g., oxidation degradation from oxygen and moisture), or to modulate the electrochemical reactions and to control the electric current generated when in use. Exemplary coating materials include inorganic or organic polymers, natural or synthetic polymers, biodegradable or bioabsorbable polymers, silica, glass, various metal oxides (e.g., oxide of zinc, aluminum, magnesium, or titanium) and other inorganic salts of low solubility (e.g., zinc phosphate). Coating methods are known in the art of metallic powder processing and metal pigment productions, such as those described in U.S. Pat. No. 5,964,936; U.S. Pat. No. 5,993,526; U.S. Pat. No. 7,172,812; US 20060042509A1 and US 20070172438.

In one embodiment, the galvanic particulates are stored in anhydrous form, e.g., as a dry powder or as an essentially anhydrous non-conducting organic solvent composition (e.g., dissolved in polyethylene glycol, propylene glycol, glycerin, liquid silicone, and/or alcohol). In another embodiment, the galvanic particulates are embedded into an anhydrous carrier (e.g., inside a polymer). In yet another embodiment, the galvanic particulates are encapsulated in compositions of microcapsules, liposomes, or micelles, or embedded in the lipophilic phase of oil-in-water (O/W) or water-in-oil (W/O) types of emulsion systems (e.g., W/O lotion, W/O ointment, or O/W creams), as well as self-emulsifying compositions, in order to achieve shelf-life stability, retard the activation of the galvanic particulates, or prolong the action of galvanic particulates.

The reaction scheme for the in situ electrochemical generation of hydrogen peroxide by galvanic particulates in the presence of water and oxygen is as follows (for example where the galvanic particulates comprise zinc and copper):

elemental zinc is oxidized at the zinc electrode surface (positive electrode) to form zinc ions:

On zinc anode (+): Zn−2e⁻→Zn²⁺

or in a general form: Metal−xe⁻→Metal^{x+} Electrochemical Reaction (1)

hydrogen ions from the water are reduced at the copper electrode (negative electrode) to form hydrogen gas:

On copper cathode (−): 2H⁺+2e⁻→H₂ Electrochemical Reaction (2)

oxygen dissolved in the water is reduced at the copper electrode to form hydrogen peroxide at low concentration:

On copper cathode (−): O₂+2e⁻+2H⁺→H₂O₂ Electrochemical Reaction (3)

### Hydrogen Peroxide Produced by Photochemical Reaction

In an alternative embodiment, the composition generates an anti-inflammatory effective amount of hydrogen peroxide in situ by photochemical reaction. In this case, the hydrogen peroxide generating agent comprises a vitamin and said composition is administered with blue, visible, or sun light.

In one embodiment, the light is administered using a Light-Emitting Diode (LED) that emits visible light at a peak wavelength of 405 nm, with a range of visible light from 395 to 415 nm.

Examples of vitamins include, but are not limited to, vitamin A, vitamin B's such as vitamin B2 (Riboflavin) vitamin B3, vitamin B5, and vitamin B12, vitamin C, vitamin K, and different forms of vitamin E including alpha, beta, gamma, or delta tocopherols or their mixtures, and derivatives thereof.

### Hydrogen Peroxide Produced by Photo-Catalytic Reaction

In another embodiment, the composition generates an anti-inflammatory effective amount of hydrogen peroxide in situ by photo-catalytic reaction. In this case, the hydrogen peroxide generating agent comprises a metal oxide, for instance in as particles or a powder, and said composition is administered with ultraviolet or visible light.

The metal oxide may comprise, for example, zinc oxide, titanium oxide, ferric oxide, or mixtures thereof. Nanometer sized metallic oxide particles, for example, nanometer-sized ZnO and TiO particles without any silicone coating, are preferred. ZnO and TiO particles spiked with small content of Fe₃O₂ or other cosmetically or therapeutically metal oxide are also preferred because of greater photo-catalytic activity.

The UV or visible light may originate from a natural light source (i.e., sun light) or a man-made light source, as known in the art.

### Hydrogen Peroxide Produced by Enzymatic Reaction

In another embodiment, the composition generates an anti-inflammatory effective amount of hydrogen peroxide in situ by enzymatic reaction. In this case, the hydrogen peroxide generating agent comprises an enzyme and its substrate.

Exemplary enzymes and their substrates include, but are not limited to, a glucose oxidase and a glucose, an amine oxidase and an amine, an amino acid oxidase and an amino acid, a lactate oxidase and a lactate, a cholesterol oxidase and a cholesterol, a uric acid oxidase and a uric acid, or a xanthine oxidase with a xanthine. Other suitable oxidases are urate oxidase, galactose oxidase, alcohol oxidase and amyloglucosidase.

### Compositions

The composition may be administered to a human or other mammal by any means used in the pharmaceutical or cosmetic arts, including topical administration, gastrointestinal (including ingestible or oral) administration, parenteral administration, nasal administration, intravaginal administration, and the like. Administration may be local or systemic.

Accordingly, the composition can be used in many consumer and medical products for human and animal applications such as in ingestible compositions (such as tablets and solutions), topical compositions (such as creams, lotions, gels, shampoos, cleansers, powders patches, bandages, and masks for application to the skin or mucosal membranes), garments (such as undergarments, underwears, bras, shirts, pants, pantyhose, socks, head caps, facial masks, gloves, and mittens), linens (such as towels, pillow covers or cases and bed sheets), and personal and medical products (such as sanitizing products for household and clinical settings, microcides for plants) and devices (such as toothbrushes, dental flosses, periodontal implants or inserts, orthodontic braces, joint wraps/supports, buccal patches, ocular inserts or implants such as contact lenses, nasal implants or inserts, and contact lens cleaning products, wound dressings, diapers, sanitary napkins, and wipes, tampons, rectal and vaginal suppositories), and coatings or embedded surfaces on medical devices and other surfaces where the anti-inflammatory effects are desired.

The compositions may alternatively be made into a wide variety of products for application on mucosal membranes, including but not limited to vaginal creams, tampons, suppositories, floss, mouthwash, or toothpaste. Other product forms can be formulated by those of ordinary skill in the art.

In one embodiment, composition of the invention is incorporated into a wound dressing or bandage.

In another embodiment, the composition is incorporated into a transdermal drug delivery patch to enhance penetration of the hydrogen peroxide generating agent into the skin by iontophoresis. In addition, such patch also reduces skin irritation by electric stimulation and electrically generated beneficial ions, such as zinc ions.

The composition may be applied directly to a target location of the body in need such a therapeutic treatment (e.g., either topically or inside the body).

### Ingestible Compositions

In one embodiment, the composition is an ingestible composition containing, per dosage unit (e.g., tablet, capsule, powder, injection, teaspoonful and the like) an amount of hydrogen peroxide generating agent necessary to deliver a substantially continuous, in situ dose as described above. In one embodiment, the ingestible compositions herein contain, per unit dosage unit, about 1 mg to about 5 g of the hydrogen peroxide generating agent, such as from about 50 mg to about 500 mg, and may be given at a dosage of from about 1 mg/kg/day to about 1 g/kg/day, such as from about 50 to about 500 mg/kg/day. The dosages, however, may be varied depending upon the requirement of the patient, the severity of the condition being treated, and the agent being employed. The use of either daily administration or post-periodic dosing may be employed. In one embodiment, these compositions are in unit dosage forms from such as tablets, pills, capsules, powders, granules, solutions or suspensions, and drops.

In one embodiment, the compositions are provided in the form of tablets, such as those containing 1, 5, 10, 25, 50, 100, 150, 200, 250, 500, and/or 1000 milligrams of the hydrogen peroxide generating agent. The composition may be administered on a regimen of 1 to 4 times per day. Advantageously, the compositions may be administered in a single daily dose, or the total daily dosage may be administered in divided doses of two, three or four times daily.

Optimal dosages to be administered may be readily determined by those skilled in the art, and will vary with the particular hydrogen peroxide generating agent used, the mode of administration, the strength of the preparation, and the advancement of the disease/condition being treated. In addition, factors associated with the particular patient being treated, including patient age, weight, diet and time of administration, will result in the need to adjust dosages.

Ingestible compositions containing one or more types of the hydrogen peroxide generating agents described herein can be prepared by intimately mixing the same with a pharmaceutically-acceptable carrier suitable for oral use according to conventional pharmaceutical compounding techniques. The carrier may take a wide variety of forms depending upon the type of formulation. Thus for liquid preparations such as suspensions, elixirs and solutions, suitable carriers and additives include but not limited to water, glycols, alcohols, silicones, waxes, flavoring agents, buffers (such as citrate buffer, phosphate buffer, lactate buffer, gluconate buffer), preservatives, stabilizers, coloring agents and the like; and for solid preparations, such as powders, capsules and tablets, suitable carriers and additives include starches, sugars, diluents, granulating agents, lubricants, binders, disintegrating agents and the like. Solid oral preparations may also be coated with substances such as sugars, soluble polymer film, insoluble-but-solute permeable polymer film. Oral preparations may also be coated with enteric coatings, which are not soluble in the acidic stomach environment but will dissolve in the intestine as the pH becomes neutral, so as to adjust the site of administration of the agent.

For preparing solid compositions such as tablets, the hydrogen peroxide generating agent is mixed with a pharmaceutically-acceptable carrier for oral use, e.g., conventional tableting ingredients such as corn starch, lactose, sucrose, sorbitol, talc, stearic acid, magnesium stearate, dicalcium phosphate or gums, and other pharmaceutically-acceptable diluents, to form a solid preformulation composition containing a homogeneous mixture. When referring to these preformulation compositions as homogeneous, it is meant that the hydrogen peroxide generating agent is dispersed evenly throughout the composition so that the composition may be readily subdivided into equally effective dosage forms such as tablets, pills and capsules. This solid preformulation composition may then subdivided into unit dosage forms of the type described above. The tablets or pills of the composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids with such materials as shellac, cetyl alcohol and cellulose acetate.

### Topical Compositions

In one embodiment, the composition is a topical composition suitable for topical administration, for example, to mammalian skin, such as human skin. In one embodiment, the composition contains a safe and effective amount of (i) the hydrogen peroxide generating agent and (ii) a pharmaceutically-acceptable or cosmetically-acceptable, topical carrier.

The composition may be made into a wide variety of products that include but are not limited to leave-on products (such as lotions, creams, gels, sticks, sprays, and ointments), skin cleansing products (such as liquid washes, solid bars, and wipes), hair products (such as shampoos, conditioners, sprays, and mousses), shaving creams, film-forming products (such as masks), make-up (such as foundations, eye liners, and eye shadows), deodorant and anti-perspirant compositions, and the like. These product types may contain several types of pharmaceutically- or cosmetically-acceptable carrier forms for topical use including, but not limited to solutions, suspensions, emulsions such as microemulsions and nanoemulsions, gels, and solids carrier forms. Other product forms can be formulated by those of ordinary skill in the art.

In one embodiment, the composition is stored in anhydrous forms, e.g., as a cosmetic powder or stick composition, or as an essentially anhydrous non-conducting organic solvent composition (e.g., dissolved or suspended in polyethylene glycols, propylene glycol, glycerin, liquid or semisolid silicones, and/or alcohol). In another embodiment, the composition is embedded into an anhydrous carrier (e.g., inside a polymer) or coated onto a substrate (e.g., as a coating or in the coating layer of a healthcare product such as wound dressing or dental floss). In yet another embodiment, composition or hydrogen peroxide generating agent is encapsulated in microcapsules, liposomes, micelles, or embedded in the lipophilic phase of oil-in-water (O/W) or water-in-oil (W/O) types of emulsion systems (e.g., W/O lotion, W/O ointment, or O/W creams), as well as self-emulsifying compositions, in order to achieve self-life stability or to prolong the action of composition.

The composition may comprise other substances, such as biologically active agents, pharmaceutical excipients, and cosmetic agents.

Additional biologically active agents include but are not limited to sunscreens, anti-wrinkling/antiaging agents, antifungal agents, antibiotic agents, anti-acne and antipsoriatic agents, depigmentating agents, where such agents may be utilized so long as they are physically and chemically compatible with the other components of the composition.

The compositions of this invention may include additional skin actives. Actives can be but not limited to vitamin compounds Skin lightening agents (kojic acid, ascorbic acid and derivatives such as ascorbyl pamiltate, and the like); anti-oxidant agents such as tocopherol and esters; metal chelators, retinoids and derivatives, moisturizing agents, hydroxy acids such as salicylic acid, sun screen such as octyl methoxycinnamate, oxybenzone, avobenzone, and the like, sun blocks such as titanium oxide and zinc oxide, and skin protectants. Mixtures of above skin actives may be used.

Sunscreens which may be used in the compositions of this invention may include but are not limited to organic or inorganic sunscreens, such as octylmethoxycinnamate and other cinnamate compounds, titanium dioxide, zinc oxide and the like.

Anti-wrinkling/anti-aging agents may include but are not limited to retinoids (for example, retinoic acid, retinol, retinal, retinyl acetate, and retinyl palmitate) alpha hydroxy acids, galactose sugars (for example, melibiose and lactose), antioxidants, including but not limited to water soluble antioxidants such as sulfhydryl compounds and their derivatives (for example, sodium metabisulfite and N-acetyl-cysteine, acetyl-cysteine), lipoic acid and dihydrolipoic acid, resveratrol, lactoferin, ascorbic acid and ascorbic acid derivatives (for example ascorbyl palmitate and ascorbyl polypeptide). Oil soluble antioxidants suitable for use in the compositions of this invention include, but are not limited to tocopherols (for example, tocopheryl acetate, α-tocopherol), tocotrienols and ubiquinone. Natural extracts containing antioxidants suitable for use in the compositions of this invention, include, but not limited to extracts containing flavonoids, phenolic compounds, flavones, flavanones, isoflavonoids, mono, di- and tri-terpenes, sterols and their derivatives. Examples of such natural extracts include grape seed, green tea, pine bark and propolis extracts and legume extracts and the like.

Antifungal agents include but are not limited to miconazole, econazole, ketoconazole, itraconazole, fluconazole, bifoconazole, terconazole, butoconazole, tioconazole, oxiconazole, sulconazole, saperconazole, clotrimazole, undecylenic acid, haloprogin, butenafine, tolnaftate, nystatin, ciclopirox olamine, terbinafine, amorolfine, naftifine, elubiol, griseofulvin, and their pharmaceutically acceptable salts.

Antibiotic (or antiseptic agents) include but are not limited to but are not limited to mupirocin, neomycin sulfate, bacitracin, polymyxin B, 1-ofloxacin, tetracyclines (chlortetracycline hydrochloride, oxytetracycline hydrochloride and tetrachcycline hydrochloride), clindamycin phosphate, gentamicin sulfate, benzalkonium chloride, benzethonium chloride, hexylresorcinol, methylbenzethonium chloride, phenol, quaternary ammonium compounds, triclocarbon, triclosan, tea tree oil, benzoyl peroxide and their pharmaceutically acceptable salts.

Acne ingredients include but are not limited to agents that normalize epidermal differentiation (e.g. retinoids), keratolytic agents (e.g. salicylic acid and alpha hydroxy acids), benzoyl peroxide, antibiotics and compounds or plant extracts that regulate sebum.

Antipsoriatic agents include but are not limited to corticosteroids (e.g., betamethasone dipropionate, betamethasone valerate, clobetasol propionate, diflrasone diacetate, halobetasol propionate, amcinonide, desoximetasone, fluocinonide, fluocinolone acetonide, halcinonide, triamcinolone acetate, hydrocortisone, hydrocortisone valerate, hydrocortisone butyrate, aclometasone dipropionte, flurandrenolide, mometasone furoate, methylprednisolone acetate), Vitamin D and its analogues (e.g. calcipotriene), retinoids (e.g. Tazarotene) and anthraline.

Cosmetic agents which may be used in the compositions of this invention may include, but are not limited to those agents which prevent potential skin irritation, such as emollients, vitamins and antioxidants (e.g., vitamin E) and herbal extracts (e.g., aloe vera). Further, the cosmetic agents may include humectants, antioxidants/preservatives, plant extracts, flavors, fragrances, surface active agents, and the like. Examples of humectants include glycerol, sorbitol, propylene glycol, ethylene glycol, 1,3-butylene glycol, polypropylene glycol, xylitol, maltitol, lactitol, oat protein, allantoin, acetamine MEA, hyaluronic acid and the like. They may be used either singly or in combination.

Cosmetic agents may also include substances which mask the symptoms of inflammatory disorders and related conditions; such substances include but are not limited to pigments, dyes, and other additives (e.g., silica, talk, zinc oxide, titanium oxide, clay powders). The pharmaceutical excipients include but are not limited to pH modifying agents such as pH-modifying agents, organic solvents (e.g., propylene glycol, glycerol, etc.), cetyl alcohol, kaolin, talc, zinc oxide, titanium oxide, cornstarch, sodium gluconate, oils (e.g., mineral oil), ceteareth-20, ceteth-2, surfactants and emulsifiers, thickener (or binders), perfume, antioxidants, preservatives, and water.

Binders or thickeners may be used in the compositions of this invention to provide substantivity and physical stability to the compositions. Binders or thickeners suitable for use in the compositions of this invention include cellulose derivatives such as alkali metal salts of carboxymethylcellulose, methyl cellulose, hydroxyethyl cellulose and sodium carboxymethylhydroxyethyl cellulose, alkali metal alginates such as sodium alginate, propylene glycol alginate, gums such as carrageenan, xanthan gum, tragacanth gum, caraya gum and gum arabic, and synthetic binders such as polyvinyl alcohol, polysodium acrylate and polyvinyl pyrrolidone. Thickeners such as natural gums and synthetic polymers, as well as coloring agents and fragrances also are commonly included in such compositions.

Examples of preservatives which may be used in the compositions of this invention include, but are not limited to, salicylic acids chlorhexidine hydrochloride, phenoxyethanol, sodium benzoate, methyl para-hydroxybenzoate, ethyl para-hydroxybenzoate, propyl para-hydroxybenzoate, butyl para-hydroxybenzoate and the like.

Examples of flavors and fragrances which may be used in the compositions of this invention include menthol, anethole, carvone, eugenol, limonene, ocimene, n-decylalcohol, citronellol, α-terpineol, methyl salicylate, methyl acetate, citronellyl acetate, cineole, linalool, ethyl linalool, vanillin, thymol, spearmint oil, peppermint oil, lemon oil, orange oil, sage oil, rosemary oil, cinnamon oil, pimento oil, cinnamon leaf oil, perilla oil, wintergreen oil, clove oil, eucalyptus oil and the like.

Examples of surface active agents which may be used in the compositions of this invention include sodium alkyl sufates, e.g., sodium lauryl sulfate and sodium myristyl sulfate, sodium N-acyl sarcosinates, e.g., sodium N-lauroyl sarcosinate and sodium N-myristoyl sarcosinate, sodium dodecylbenzenesulfonate, sodium hydrogenated coconut fatty acid monoglyceride sulfate, sodium lauryl sulfoacetate and N-acyl glutamates, e.g., N-palmitoyl glutamate, N-methylacyltaurin sodium salt, N-methylacylalanine sodium salt, sodium a-olefin sulfonate and sodium dioctylsulfosuccinate; N-alkylaminoglycerols, e.g., N-lauryldiaminoethylglyecerol and N-myristyldiaminoethylglycerol,N-alkyl-N-carboxymethylammonium betaine and sodium 2-alkyl-1-hydroxyethylimidazoline betaine; polyoxyethylenealkyl ether, polyoxyethylenealkylaryl ether, polyoxyethylenelanolin alcohol, polyoxyethyleneglyceryl monoaliphatic acid ester, polyoxyethylenesorbitol aliphatic acid ester, polyoxyethylene aliphatic acid ester, higher aliphatic acid glycerol ester, sorbitan aliphatic acid ester, Pluronic type surface active agent, and polyoxyethylenesorbitan aliphatic acid esters such as polyoxyethylenesorbitan monooleate and polyoxyethylenesorbitan monolaurate. Emulsifier-type surfactants know to those of skill in the art should be used in the compositions of this invention.

Another important ingredient of the present invention is a dermatologically acceptable, topical carrier. It is not only compatible with the active ingredients described herein, but will not introduce any toxicity and safety issues. An effective and safe carrier varies from about 50% to about 99% by weight of the compositions of this invention, more preferably from about 75% to about 99% of the compositions and most preferably from about 85% to about 95% by weight of the compositions.

The choice of which pharmaceutical excipient or biological agent, or cosmetic agent to use is often controlled or affected by the type of inflammatory disorder or related condition which is being treated. For example, if the compositions of this invention were used to treat a skin inflammation associated with athlete's foot, jock itch or diaper rash, talc would be a preferred pharmaceutical excipient and an antifungal agents would be preferred biological agents. If the compositions of this invention were to be used to treat eczema of the scalp, emulsifiers and oils would be preferred pharmaceutical excipients.

The following non-limiting examples further illustrate the invention.

### Example 1

### Reduction in Pro-Inflammatory Mediator Release with Sustained Low Level of Hydrogen Peroxide

NHEK (Normal Human Epidermal Keratinocytes) cells were seeded into a 96-well plate at 1.5×10⁴ cells/well, 48 hours later the wells were 85% confluent. The keratinocyte cells were then treated with and without exposure to 6×10⁵ P. Acnes cells/well and in the presence or absence of various concentrations of Hydrogen peroxide: 10 μM, 100 μM, and 1000 μM.

Eighteen hours after the treatments with H₂O₂ the supernatant media was collected from each well and analyzed for the levels of IL-1a and IL-8 cytokines, pro-inflammatory markers, produced by the NHEK cells using a commercially available cytokine detection kit (Upstate Biotechnology, Charlottesville, Va.). The results are shown in Table 1.

TABLE 1

Cytokine Release

| | IL-1RA | Percent (%) |
|---|---|---|
| Treatment | (pmol/ml) | Reduction |
| Unstimulated | 179.1 ± 74.2 | — |
| P. acnes Stimulated | 670.1 ± 37.1 | — |
| P. acnes + H2O2 (10 μM) | 624.1 ± 94.5 | 6.8% |
| P. acnes + H2O2 (100 μM) | 327.2 ± 88.3 | 51.1% |
| P. acnes + H2O2 (1000 μM) | 206.7 ± 60.23 | 69.1% |

The data in Table 1 shows that low levels of H₂O added to human keratinocytes reduced the level of pro-inflammatory markers generated by the cells when exposed to the bacteria P. acnes, an important factor in the development of inflammatory acne lesions.

### Example 2

### Production of Hydrogen Peroxide and Subsequent Reduction of Pro-Inflammatory Mediator Release with Galvanic Particulates

NHEK cells were seeded into a 96-well plate at 1.5×10⁴ cells/well, 48 hours later the wells were 85% confluent. The NHEK cells were stimulated by exposure to 6×10⁵ P. Acnes cells/well in the presence or absence of 50 μg/ml galvanic particulate following the methods of Sur et al., J Invest Dermatol. 2008 128(2):336-344). The galvanic particulates comprised zinc-copper bimetallic particles produced using an electroless deposition method using fine zinc metal (99.996% purity) and a copper salt solution. The galvanic particles comprised a zinc core with copper deposited thereon, both metals being exposed on the surface. The particles had an average particle size of 22 μm and had a galvanic current in the range of 70-90 μA/mg according to the method of V. Ligier et al., “Formation of the Main Atomospheric Zinc End Products: NaZn₄(OH)₆.6H₂O, Zn₄SO₄(OH)₆.nH₂O and Zn₄Cl₂(OH)₄SO₄.5H₂O in [C⁻][SO₄²⁻[HCO₃⁻[H₂O₂] Electrolytes”. Corrosion Science 1999; 41:1139-1164. Cells were treated with the galvanic particulates for 1 hour.

A portion of the cells treated with the galvanic particulates were additionally treated with the enzyme catalase (20 units/ml), which is known to break down H₂O₂.

Following 24 hour incubation at 37° C. with 5% CO₂, supernatants were removed and analyzed for the levels of IL-1A cytokine in the manner of Example 1.

To detect hydrogen peroxide production, the keratinocytes were loaded for a 30-minute incubation period with 5 μM of the hydrogen peroxide-sensitive fluorescent probe 5-(and-6)-chloromethyl-2′,7′-dichlorodihydrofluorescein diacetate, acetyl ester (CM-H2DCFDA, Invitrogen Carlsbad, Calif.). Hydrogen peroxide production was quantitated using a fluorescent plate reader set at wavelengths 485 excitation/530 emission.

The results are shown in Table 2.

TABLE 2

| | | | Hydrogen |
|---|---|---|---|
| | IL-1A | Percent (%) | peroxide |
| Treatment | (pmol/ml) | Reduction | Formation (μM) |
| Unstimulated | 193.4 ± 14.2 | — | 0.05 ± 0.01 |
| P. acnes Stimulated | 578.2 ± 37.5 | — | 0.06 ± 0.02 |
| P. acnes + Galvanic | 269.1 ± 8.6 | 46.5% | 1.153 ± 0.13 |
| particulates (50 μg/ml) | | | |
| P. acnes + Galvanic | 477.3 ± 50.4 | 17.5% | 0.23 ± 0.01 |
| particulates (50 μg/ml) + | | | |
| Catalase (20 units/ml) | | | |

The data in Table 2 shows that the galvanic particulates produced hydrogen peroxide and reduced the level of the pro-inflammatory mediator IL-1A induced by bacterial stimulation. Furthermore, the anti-inflammatory activity of the galvanic particulates was reversed by the addition of catalase. Taken together, the data indicates that the anti-inflammatory activity of the galvanic particulates was generated, at least in part, by the production of hydrogen peroxide.

### Example 3

### Topical Anti-Inflammatory Activity in a Murine Model of Contact Hypersensitivity

The ability of topically applied of galvanic particulates or hydrogen peroxide to affect inflammatory response was demonstrated using in an in vivo immune cell-mediated skin inflammation model as follows.

Albino male CD-1 mice, 7-9 weeks old, were induced on the shaved abdomen with 50 μl of 3% oxazolone in acetone/corn oil (Day 0). On Day 5, a 20 μl volume of 2% oxazolone in acetone was applied to the dorsal left ear of the mouse. Galvanic particulates (as described in Example 2) or H₂O₂ was then applied to the left ear in a volume of 20 μl one hour after oxazolone challenge in a 70% ethanol/30% propylene glycol vehicle. The right ear was not treated. The mice were sacrificed by CO₂ inhalation 24 hours after the oxazolone challenge, the left and right ears were removed and a 7-mm biopsy was taken from each ear and weighed. The difference in biopsy weights between the right and left ear was calculated.

Anti-inflammatory effects were determined as an inhibition of the increase in ear weight. Application of 1 mg/ml of hydrocortisone, a known anti-inflammatory compound, was used as a positive control. The results are shown in Table 3, where the Percent Inhibition of Skin Inflammation was calculated as (Vehicle treated biopsy weight−Agent(s) treated biopsy weight)/(Vehicle treated biopsy weight)×100.

TABLE 3

| Treatment (Dose) | Percent Inhibition of Skin Inflammation |
|---|---|
| Hydrocortisone (1 mg/ml) | 70.3% ± 6.6% |
| Galvanic particulates (1 mg/ml) | 60.4% ± 9.6% |
| Hydrogen Peroxide (0.001% or | 66.5% ± 8.3% |
| 10 ug/ml) | |
| Hydrogen Peroxide (0.0001% or | 73.0% ± 9.4% |
| 1 ug/ml) | |

The data in Table 3 shows that the topical application of galvanic particulates demonstrated anti-inflammatory activity comparable to a corticosteroid. The low level hydrogen peroxide added separately produced a similar degree of anti-inflammatory activity.

### Example 4

### Production of Hydrogen Peroxide and Subsequent Reduction in Pro-Inflammatory Mediator Release with Light Emitting Diode and Vitamin Combination

The ability of a combination of a Light-Emitting Diode (LED) containing device that emits visible light at a peak wavelength of 405 nm, with a range of visible light from 395 to 415 nm and the vitamin B2 (Riboflavin) (Sigma Aldrich, St Louis, Mo.) to synergistically generate low level hydrogen peroxide and produce an anti-inflammatory effect was illustrated by its ability to reduce macrophage activation in the following assay.

Murine macrophage cell line (RAW264) cells were adjusted to a density of 4×10⁶ cells/mL in DMEM with 10% FBS (American Type Culture Collection, Manassas, Va.) and 100 μl was added to a flat-bottomed 96-well tissue culture plate. Cells were exposed to LED light alone (5 minutes exposure), riboflavin alone, or a combination of LED light (5 minutes exposure) and riboflavin, 30 minutes prior to stimulation 0.1 μg/ml of the bacterial cell well component, lipopolysaccharide (LPS), for 24 hours at 37° C. with 5% CO₂. The cytokine IL-1A, a potent pro-inflammatory mediator, were assayed for with using an ELISA assay.

The results are shown in Table 4. Results are expressed as the percent inhibition of inflammatory mediator production compared to a stimulated control culture. Hydrogen peroxide levels were determined using the method described in Example 2.

TABLE 4

| | | | Hydrogen |
|---|---|---|---|
| | IL-1A | Percent (%) | peroxide |
| Treatment | (pmol/ml) | Reduction | Formation (μM) |
| Unstimulated | 0.11 ± 0.03 | — | 0.02 ± 0.01 |
| LPS Stimulated | 0.557 ± 0.057 | — | 0.05 ± 0.02 |
| LPS + Light Alone | 0.574 ± 0.024 | −3.8% | 0.08 ± 0.03 |
| LPS + Riboflavin | 0.551 ± 0.058 | 11.5% | 0.05 ± 0.01 |
| Alone | | | |
| (10000 ng/ml) | | | |
| LPS + Light + | 0.453 ± 0.027 | 23.2% | 0.367 ± 0.14 |
| Riboflavin | | | |
| (10 ng/ml) | | | |
| LPS + Light + | 0.277 ± 0.020 | 62.6% | 0.451 ± 0.09 |
| Riboflavin | | | |
| (100 ng/ml) | | | |
| LPS + Light + | 0.103 ± 0.005 | 101.5% | 0.557 ± 0.10 |
| Riboflavin | | | |
| (1000 ng/ml) | | | |
| LPS + Light + | 0.097 ± 0.002 | 102.8% | 0.968 ± 0.21 |
| Riboflavin | | | |
| (10000 ng/ml) | | | |

The data in Table 4 shows that vitamin B2 administered with light increased hydrogen peroxide production and modulated the release of pro-inflammatory mediators induced by bacterial protein (LPS) stimulation.

### Example 5

### Production of Hydrogen Peroxide by Enzymatic Reaction of Glucose and Glucose Oxidase

Human keratinocyte cells were seeded in an assay plate at identical densities and incubated for 48 hours at 37° C. with 5% CO₂. The cells were treated with glucose alone, glucose oxidase alone, or a combination of the two for 1 hour. Treatment of control wells with 0.01% hydrogen peroxide served as a positive control. The results are shown in Table 5.

TABLE 5

| | Peroxide Formation (μM) After 60 |
|---|---|
| Treatment | Minutes |
| Untreated | 0.11 ± 0.03 |
| Glucose Only (50 μM) | 0.34 ± 0.04 |
| Glucose Oxidase (0.1 unit/ml) | 0.37 ± 0.03 |
| Glucose (50 μM) + Glucose | 20.56 ± 1.09** |
| Oxidase (0.1 unit/ml) | |
| H₂O₂ (0.01%) | 6.51 ± 1.23** |

**Indicates significant difference from untreated peroxide formation at 60 minutes using a student's t-Test with significance set at P < 0.05.

Hydrogen peroxide production in the glucose plus glucose oxidase treated cells was measured over 90 minutes using the method described in Example 2. The results are shown in Table 6.

TABLE 6

| | Hydrogen Peroxide Production (μM) | | | | | | |
|---|---|---|---|---|---|---|---|
| | | 15 | 30 | 45 | 60 | 75 | 90 |
| Treatment | Baseline | Minutes | Minutes | Minutes | Minutes | Minutes | Minutes |
| Glucose + | 0.15 ± 0.12 | 7.0 ± 0.56** | 12.57 ± 0.76** | 16.86 ± 0.93** | 20.56 ± 1.09** | 23.52 ± 1.02** | 25.20 ± 0.19** |
| Glucose Oxidase | | | | | | | |

**Indicates significant difference from baseline hydrogen peroxide production at that timepoint using a student's t-Test with significance set at P < 0.05.

The data in Tables 5 and 6 show that treatment with the combination of glucose oxidase and its substrate, glucose, produced hydrogen peroxide on a continuous basis. The amount of hydrogen peroxide generated by the combination was substantially greater than that produced by glucose oxidase or glucose alone. Therefore, the combination is expected to provide an effective anti-inflammatory benefit.

### Example 6

### Inhibition of NF-kB Activation

Nuclear Factor Kappa Beta (NF-kB) is a transcription factor that binds to the NF-kB binding site on the promoter region of pro-inflammatory genes, such as COX-2 and Nitric Oxide Synthase (iNOS) (Bell S, et al (2003) Cell Signal.; 15(1):1-7). NF-kB is involved in regulating many aspects of cellular activity, in stress, injury and especially in pathways of the immune response by stimulating synthesis of pro-inflammatory proteins, such as cycloxygenase-2 (COX-2), thus leading to inflammation (Chun K S, et al. (2004) Carcinogenesis 25:445-454.; Fenton M J (1992) Int J Immunopharmacol 14:401-411). NF-kB itself is induced by stimuli such as pro-inflammatory cytokines (e.g. TNF-alpha and IL-1beta), bacterial toxins (e.g. LPS and exotoxin B), a number of viruses/viral products (e.g. HIV-1, HTLV-I, HBV, EBV, and Herpes simplex), as well as pro-apoptotic and necrotic stimuli (e.g., oxygen free radicals, UV light, and gamma-irradiation) Inhibition of NF-kB activation is likely to reduce inflammation by blocking the subsequent signaling that results in transcription of new pro-inflammatory genes.

Solar ultraviolet irradiation activates the transcription factor NF-kB, inducing the production of matrix metalloproteinases that can lead to degradation of matrix proteins such as elastin and collagen. Inhibitors of NF-kB are likely to inhibit the subsequent signaling that results in the presence of MMPs in the dermal matrix, and the more of the pathway that is inhibited, the more likely there will be no induction of MMPs. Recently inhibition of the NF-kB pathway has shown to result in a subsequent induction in collagen synthesis (Schreiber J, et al. (2005) Surgery. 138:940-946). Thus, inhibition of NF-kB activation may also provide anti-aging benefits to skin by increasing collagen synthesis.

The activity of galvanic particulates as described in Example 2 in blocking NF-kB activation was studied as follows. FB293 cells, a stable transfected human epithelial cell line containing the gene reporter for NF-kB obtained from Panomics (Fremont, Calif.), were used. FB293 cells were plated at a density of 5×10⁴ cells/mL in Dulbecco's modified Eagle's medium (DMEM) supplemented with 10% fetal bovine serum (Invitrogen, San Diego, Calif.). FB293 cells were stimulated with 50 ng/mL 12-O-tetradecanoylphorbol 13-acetate (TPA) (Sigma St Louis, Mo.) in the presence or absence of galvanic particulates. Following a 24 hour incubation at 37° C. with 5% CO2, cells were lysed with 40 μl of reporter lysis buffer (Promega, Madison, Wis.). A 20-μl aliquot of the lysate was assayed using a luciferase assay kit (Promega) and counted for 10 seconds in a Lmax luminometer (Molecular Devices, Sunnyvale, Calif.) with the data represented as the relative light unit/second. Galvanic particulates were found to inhibit NF-kB activation as shown in Table 7.

TABLE 7

| | NF-kB Gene Reporter | |
|---|---|---|
| | Activation | Percent |
| | (Luminescence) | Inhibition |
| Untreated | 4.06 ± 0.6 | — |
| TPA (10 ng/ml) Stimulated | 28.46 ± 2.21 | — |
| TPA + Galvanic particulates | 3.20 ± 1.98 | 88.7% |
| (100 ug/ml) | | |
| UV (10 kJ) Stimulated | 11.45 ± 1.89 | — |
| UV (10 kJ) + Galvanic | 5.51 ± 1.74 | 51.6% |
| particulates (100 ug/ml) | | |

Galvanic particulates, thus, were found to substantially reduce NF-kB activation. This example demonstrates that galvanic particulates can modulate the production of inflammatory mediators, which contribute to inflammation of the skin. This example also demonstrates that galvanic particulates may also protect elastin and collagen fibers from damage and degradation that can lead to aging of the skin.

### Example 7

### Anti-Inflammatory Activity on Release of Uv-Induced Pro-Inflammatory Mediators on Reconstituted Epidermis

The effect of galvanic particulates was evaluated for topical anti-inflammatory activity on human epidermal equivalents. Epidermal equivalents (EPI 200 HCF), multilayer and differentiated epidermis consisting of normal human epidermal keratinocytes, were purchased from MatTek (Ashland, Mass.). These epidermal equivalents were incubated for 24 hours at 37° C. in maintenance medium without hydrocortisone. Equivalents were topically treated (2 mg/cm2) with galvanic particulates (1 mg/ml) from Example 1(a) in 70% ethanol/30% propylene glycol vehicle 2 hours before exposure to solar ultraviolet light (1000W-Oriel solar simulator equipped with a 1-mm Schott WG 320 filter; UV dose applied: 70 kJ/m2 as measured at 360 nm). Equivalents were incubated for 24 hours at 37° C. with maintenance medium then supernatants were analyzed for IL-8 cytokine release using commercially available kits (Upstate Biotechnology, Charlottesville, Va.). The results are depicted in Table 8.

TABLE 8

| | Mean +/− Std Dev | Percent |
|---|---|---|
| Treatment | of IL-1A | Inhibition of Skin |
| (Dose, as % w/v) | Release (ng/ml) | Inflammation |
| Untreated, No UV | 223.5 ± 168.0 | — |
| UV (60 KJ), Vehicle | 944.9 ± 205.3 | — |
| Treated | | |
| UV (60 KJ) + Galvanic | 477.7 ± 177.9** | 50.4% |
| particulates (1 mg/ml) | | |

**Indicates significant difference from UV, Vehicle treated using a student's t-Test with significance set at P < 0.05.

Based on this example, topical application of galvanic particulates was able to significantly reduce the UV-stimulated release of inflammatory mediators. Therefore, galvanic particulates would be expected to provide an effective the anti-inflammatory benefit when applied to skin.

### Example 8

### Reduction of Methyl Nicotinate-Induced Skin Erythema

Methyl nicotinate (methyl 3-pyridinecarboxylate) is a known vasodilator causing an increased cutaneous blood flow upon its application on the skin. See, Guy R. H., Arch. Dermatol Res (1982) 273:91-95. In this experiment, between 10 mM-solution of methyl nicotinate (Aldrich Chemical, St. Louis, Mo.) was topically applied for 30 seconds under occlusion (2.5 cm disk, Hill Top Research Inc, Cincinnati, Ohio) on the volar forearm of volunteers based on the method of Jumbelic et al. (Skin Pharmacol Physiol. (2006) 19:147-152). Galvanic particulates (10 mg/ml) as described in Example 2 in 70% ethanol/30% propylene glycol vehicle were topically applied after induction of erythema by methyl nicotinate challenge. Redness was assessed by diffuse reflectance spectroscopy. See Kollias N, et al., Photochem Photobiol. (1992) (56):223-227. An Ocean Optics diode array spectrophotometer (Dunedin, Fla.) connected to a HP laptop computer through a USB port was used to control the experiment and to collect and analyze the spectral data.

An optic fiber bundle was used to conduct the light from the lamp to the skin and transmit the reflectance measurements back from the skin to the spectrophotometer. The results are depicted in Table 9.

TABLE 9

| | Mean +/− Std Dev | Percent |
|---|---|---|
| Treatment | of Apparent | Inhibition of Skin |
| (Dose, as % w/v) | Hemoglobin | Erythema |
| Placebo | 0.72 ± 0.22 | — |
| Galvanic particulates | 0.43 ± 0.19 ** | 40.2% |
| (10 mg/ml) | | |

** Indicates significant difference from Placebo treated using a student's t-Test with significance set at P < 0.05.

These results indicate that topical application of galvanic particlulates reduced the erythema on a methyl nicotinate-induced human redness model.

### Example 9

### Reversal of NF-kB Inhibition of Tropoelastin Formation

The ability of galvanic particulates as described in Example 2 to reverse a TNFa-induced decrease in tropoelastin formation was demonstrated as follows.

Cultures of cardiac myoblasts H9C2 cells were transiently transfected with the elastin promoter-luciferase reporter construct (E1p2.2, a 2.2 kb elastin promoter fragment from nt −2267 to nt +2, driving the firefly luciferase gene, which was obtained from Promega, Madison Wis.). DNA was prepared by Qiagen Maxi columns (Qiagen Valencia, Calif.). In all transfections, a construct with the thymidine kinase promoter and the Renilla luciferase reporter gene (pRL-TK, Promega, Madison Wis.) was included as an internal control. Typically, cells grown in 48-well plates were transfected with 0.45 ug total DNA per well using Lipofectamine 2000 (Invitrogen life technologies, Carlsbad, Calif.). One day after transfection, cells were treated with galvanic particulates at indicated concentrations for approximately 24 hours before they were lysed for luciferase assays, using Dual-Luciferase Reporter System from Promega (Madison, Wis.), following manufacturer's protocol. Briefly, the firefly luciferase activity was measured first (representing elastin promoter activity), followed by the renilla luciferase (internal control), using luminometer LMAX, from Molecular Devices (Sunnyvale, Calif.). The ratio of these two luciferase activities (RLU) was used to evaluate the activity of each promoter.

The results are shown in Table 10.

TABLE 10

| | Tropoelastin Promoter | Fold increase Over |
|---|---|---|
| | Activity (Firefly/Renilla | Vehicle + TNF-α |
| Treatment | Luciferase: RLU) | (100 ng/mL) |
| Vehicle + TNF-α | 1.365 | — |
| (100 ng/mL) | | |
| Galvanic | 3.499 | 2.56* |
| Pacticulates | | |
| (1 μg/mL) + | | |
| TNF-α | | |
| (100 ng/mL) | | |
| Galvanic Particulates | 3.163 | 2.32* |
| (10 μg/mL) + TNF-α | | |
| (100 ng/mL) | | |

*p < 0.05 compared to vehicle (Student's T-test)

The data in Table 10 shows the galvanic particulates were able to restore the formation of tropoelastin, which was repressed by subclinical inflammation. Using galvanic particulates, which were shown in Example 6 to block NF-kB, this data shows that a TNFa-induced decrease in tropoelastin formation was reversed. Thus galvanic particulates can block the subclinical inflammation effects on aging.

### Example 10

### Continuous Production of Hydrogen Peroxide Production by Galvanic Particulates

The ability of galvanic particulates to produce hydrogen peroxide continuously in situ was illustrated in the following assay.

Human keratinocyte cells were seeded in an assay plate at identical densities and incubated for 48 hours at 37° C. with 5% CO₂. A 1% solution of Galvanic particulates was prepared in cell culture medium and 200 μL of the solution was applied to the keratinocytes over a period of 240 minutes.

Cells were treated with a 1% galvanic particulates solution in cell media over a period of 240 minutes. Treatment of control wells with 0.03% hydrogen peroxide served as a positive control.

The results are shown in Table 11.

TABLE 11

| | | 30 | 60 | 200 | 240 |
|---|---|---|---|---|---|
| Treatment | Baseline | Minutes | Minutes | Minutes | Minutes |
| Untreated | 42.3 ± | 61.4 ± | 88.1 ± | 215.4 ± | 243.9 ± |
| | 9.3 | 13.9 | 29.5 | 125.8 | 138.9 |
| Galvanic | 77.3 ± | 385.5 ± | 726.6 ± | 877.6 ± | 842.2 ± |
| particulates | 16.2 | 98.6** | 158.6** | 186.3** | 176.2** |
| (1%) | | | | | |

**Indicates significant difference from baseline hydrogen peroxide levels at that timepoint using a student's t-Test with significance set at P < 0.05.

### Example 11

### Immunomodulation of Human T-Cell Cytokine Release Stimulated with PHA

The ability of the galvanic particulates as described in Example 2 to modulate immune responses was illustrated by their ability to reduce the production of cytokines by activated human T-cells stimulated with the T-cell receptor (TCR) activating agent phytohaemagglutinin (PHA) in the following assay.

Human T-cells were collected from a healthy adult male via leukopheresis. The T-cells were isolated from peripheral blood via Ficol gradient, and the cells were adjusted to a density of 1×10⁶ cells/mL in serum free lymphocyte growth medium (ExVivo-15, Biowhittaker, Walkersville, Md.). Human T-cells were stimulated with 10 μg/mL PHA in the presence or absence of test compounds following published method (Hamamoto Y., et al. Exp Dermatol 2:231-235, 1993). Following a 48 hour incubation at 37° C. with 5% CO₂, supernatant was removed and evaluated for cytokine content using commercially available multiplex cytokine detection kit. The results are depicted in Table 12.

TABLE 12

| | Cytokine Release | |
|---|---|---|
| | | Percent (%) |
| Treatment | IL-2 (pmol/ml) | Reduction |
| Unstimulated | 2.8 ± 4.0 | — |
| PHA Stimulated | 563.2 ± 60.0 | — |
| PHA + Copper Metal (100 ug/ml) | 498.9 ± 64.4 | 11.4% |
| PHA + Zinc Metal (100 ug/ml) | 456.8 ± 11.1 | 18.9% |
| PHA + Zinc Chloride (100 ug/ml) | 566.3 ± 20.6 | −0.6% |
| PHA + Copper (II) Acetate (100 ug/ml) | 312.9 ± 96.8 | 44.4% |
| PHA + Galvanic particulates (100 ug/ml) | 10.15 ± 3.5 | 98.2% |
| Hydrocortisone (Pos. Control 100 ug/ml) | 7.69 ± 5.64 | 98.6% |

(where IL-2 = Interleukin-2 (Cytokine)).

The galvanic particulates were found to be able to modulate the release of inflammatory mediators induced by T-cell stimulation. Furthermore, the anti-inflammatory activity was greater than that of copper metal powder, zinc metal powder, copper ion (Copper (II) Acetate), or zinc ions (Zinc Chloride) alone.

### Example 12

### Immunomodulation of Human T-Cell Cytokine Release Stimulated with PHA Using Galvanic Particulates and its Water Supernatant

The ability of galvanic particulates and its water supernatant to modulate immune responses was illustrated by its ability to reduce the production of cytokines by activated human T-cells stimulated with the T-cell receptor (TCR) activating agent phytohaemagglutinin (PHA) in the following assay.

Galvanic particulates (100 μg/ml) were prepared as a suspension in deinoized water. After 1 hour the supernatant was taken and centrifuged to remove the galvanic particulates, then the water phase was taken and exposed to activated human t-cells as described below. Human T-cells were collected, isolated and tested as described in Example 11. The results are shown in Table 13.

TABLE 13

| | Cytokine Release | |
|---|---|---|
| | | Percent (%) |
| Treatment | IL-2 (pmol/ml) | Reduction |
| Unstimulated | 4.6 ± 1.2 | — |
| PHA Stimulated | 847.5 ± 45.1 | — |
| PHA + Galvanic particulates (100 μg/ml) | 22.8 ± 3.2 | 97.3% |
| PHA + Water Supernatent from Galvanic | 45.7 ± 19.6 | 94.5% |
| particulates (100 μg/ml) Solution | | |
| PHA + Hydrocortisone (Pos. Control 100 | 13.3 ± 7.2 | 98.4% |
| μg/ml) | | |

where IL = Interleukin (Cytokine)

The date in Table 13 shows that both the galvanic particulates and the water supernatent from a 100 μg/ml solution thereof were able to modulate the release of inflammatory mediators induced by T-cell stimulation. The water supernatent had been centrifuged to remove any galvanic particulates, thus only products generated by the galvanic particulates were present in the water supernatant fraction.

Furthermore, since it has been demonstrated herein that hydrogen peroxide produced by galvanic particulates can inhibit NF-KB, these results demonstrate that 1 hour of continuous in situ production hydrogen peroxide is sufficient to inhibit subclinical inflammation and may restore matrix protein expression in aging skin.

1. A method of treating inflammation in a mammalian tissue comprising administering to said tissue a composition capable of substantially continuously generating an anti-inflammatory effective amount of hydrogen peroxide in situ.

2. The method of claim 1, wherein said inflammation is sub-clinical inflammation.

3. The method of claim 1, wherein said inflammation is clinical inflammation.

4. The method of claim 1, wherein said composition comprises a hydrogen peroxide generating agent selected from the group consisting of galvanic particulates, vitamins, metal oxides, enzymes and their substrates, and combinations thereof.

5. The method of claim 1 wherein said composition comprises galvanic particulates.

6. The method of claim 5, wherein said galvanic particulates comprise a first conductive material and a second conductive material, wherein both said first conductive material and said second conductive material are exposed on the surface of said galvanic particulates, the particle size of said galvanic particulates is from about 10 nanometers to about 100 micrometers, and the difference in Standard Potentials of said first conductive material and said second conductive material is at least about 0.2 V.

7. The method of claim 6, wherein said galvanic particulates comprise said first conductive material partially coated with said second conductive material.

8. The method of claim 5, wherein said galvanic particulates comprise at least 95 percent, by weight, of said first conductive material and said second conductive material.

9. The method of claim 5, wherein said first conductive material is zinc.

10. The method of claim 5, wherein said second conductive material is copper or silver.

11. The method of claim 1, wherein said composition comprises at least one vitamin and said composition is administered with blue, visible, or sun light.

12. The method of claim 1, wherein said composition comprises at least one metal oxide and said composition is administered with ultraviolet or visible light.

13. The method of claim 1, wherein said composition comprises at least one enzyme and its substrate.

14. The method of claim 1, wherein said anti-inflammatory effective amount is less than about 0.5% by weight of the composition.

15. The method of claim 1, wherein said anti-inflammatory effective amount is about 0.0001 to about 0.5% by weight of the composition.

16. The method claim 1, wherein said tissue is selected from the group consisting of epidermis and epithelial tissue.

17. A method of substantially continuously generating hydrogen peroxide in situ on a mammalian tissue, which comprises contacting said tissue with a composition comprising a hydrogen peroxide generating agent selected from the group consisting of galvanic particulates, vitamins, metal oxides, enzymes and their substrates, and combinations thereof.

18. The method of claim 17, wherein said composition comprises galvanic particulates that comprise a first conductive material and a second conductive material, wherein both said first conductive material and said second conductive material are exposed on the surface of said galvanic particulates, the particle size of said galvanic particulates is from about 10 nanometers to about 100 micrometers, and the difference in Standard Potentials of said first conductive material and said second conductive material is at least about 0.2 V.

19. The method of claim 18, wherein said galvanic particulates comprise said first conductive material partially coated with said second conductive material.

20. The method of claim 18, wherein said galvanic particulates comprise at least 95 percent, by weight, of said first conductive material and said second conductive material.

21. The method of claim 18, wherein said first conductive material is zinc.

22. The method of claim 18, wherein said second conductive material is copper or silver.

23. The method of claim 17, wherein said composition comprises at least one vitamin and said composition is administered with blue, visible, or sun light.

24. The method of claim 17, wherein said composition comprises at least one metal oxide and said composition is administered with ultraviolet or visible light.

25. The method of claim 17, wherein said composition comprises at least one enzyme and its substrate.

26. The method claim 17, wherein said tissue is selected from the group consisting of epidermis and epithelial tissue.

27. An anti-inflammatory composition capable of substantially continuously generating an anti-inflammatory effective amount of hydrogen peroxide in mammalian tissues in situ.

28. The composition of claim 27 comprising a carrier suitable for topical administration.

29. The composition of claim 27 comprising a carrier suitable for gastrointestinal administration.

30. The composition of claim 27 comprising a hydrogen peroxide generating agent selected from the group consisting of galvanic particulates, vitamins, metal oxides, enzymes and their substrates, and combinations thereof.

31. The composition of claim 27 comprising galvanic particulates.

32. The composition of claim 31, wherein said galvanic particulates comprise a first conductive material and a second conductive material, wherein both said first conductive material and said second conductive material are exposed on the surface of said galvanic particulates, the particle size of said galvanic particulates is from about 10 nanometers to about 100 micrometers, and the difference in Standard Potentials of the first conductive material and the second conductive material is at least about 0.2 V.

33. The composition of claim 32, wherein said galvanic particulates comprise said first conductive material partially coated with said second conductive material.

34. The composition of claim 31, wherein said galvanic particulates comprise at least 95 percent, by weight, of said first conductive material and said second conductive material.

35. The composition of claim 31, wherein said first conductive material is zinc.

36. The composition of claim 31, wherein said second conductive material is copper or silver.

37. The composition of claim 27 comprising at least one vitamin, wherein said composition is administered with blue, visible, or sun light.

38. The composition of claim 27 comprising at least one metal oxide, wherein said composition is administered with ultraviolet or visible light.

39. The composition of claim 27 comprising at least one enzyme and its substrate.

40. The composition of claim 27, wherein said anti-inflammatory effective amount is less than about 0.5% by weight of the composition.

41. The composition of claim 27, wherein said anti-inflammatory effective amount is about 0.0001 to about 0.5% by weight of the composition.
